(19) United States
(12) Patent Application Publication
(10) Pub. No.: US 2012/0071577 A1
(43) Pub. Date: Mar. 22, 2012

(54) USE OF ISOCYANATES BASED ON RENEWABLE RAW MATERIALS

(75) Inventors: Jan Pfeffer, Essen (DE); Martina Ortelt, Duelmen (DE); Emmanouil Spyrou, Schermbeck (DE); Thomas Haas, Muenster (DE); Uwe Korek, Duelmen (DE); Harald Schmidt, Muelheim an der Ruhr (DE); Uwe Dingerdissen, Seeheim (DE)

(73) Assignee: EVONIK DEGUSSA GmbH, Essen (DE)

(21) Appl. No.: 13/375,664

(22) PCT Filed: Apr. 23, 2010
(86) PCT No.: PCT/EP2010/055422

§ 371 Date: Dec. 1, 2011

(30) Foreign Application Priority Data
Jul. 1, 2009 (DE) 102009027394.8

(51) Int. Cl. C09D 175/04; C08G 18/02; C09D 175/00; C08G 18/77; C08G 18/62; C08J 9/00; C08G 18/58; C09D 163/00; C09D 175/12; C09D 129/04; C09D 133/14; C08G 18/42; C08G 18/83; C08G 18/44; C08G 18/48; C07D 493/04

(52) U.S. Cl. 521/141; 549/464; 521/161; 524/589; 528/73; 528/45; 528/67; 521/160; 524/590; 528/68; 521/149; 521/156; 523/400; 524/557; 524/558; 525/56; 525/123; 525/418; 525/457; 525/467

(57) ABSTRACT

The invention relates to the use of 2,5-diisocyanato-1,4:3,6-dianhydro-2,5-dideoxy-D-manitol (I), 2,5-diisocyanato-1,4:3,6-dianhydro-2,5-dideoxy-D-glucitol (II), and/or 2,5-diisocyanato-1,4:3,6-dianhydro-2,5-dideoxy-L-iditol (III).

The invention relates to the use of 2,5-diisocyanato-1,4:3,6-dianhydro-2,5-dideoxy-D-mannitol (I), 2,5-diisocyanato-1,4:3,6-dianhydro-2,5-dideoxy-D-glucitol (II) and/or 2,5-diisocyanato-1,4:3,6-dianhydro-2,5-dideoxy-L-iditol (III).

Renewable raw materials are products of agriculture and forestry that are not used as a feed- or foodstuff. They are exploited for their substance, but also for the generation of heat, electricity or fuels.

Polyurethanes formed from customary isocyanates known in polyurethane chemistry and from 1:4-3:6 dianhydrohexitols are known from U.S. Pat. No. 4,443,563 and DE-A 31 11 093. A disadvantage is the restriction to dianhydrohexitols, lacking the breadth of variation of nowadays-customary polyols and polyol mixtures.

Likewise known are polyurethanes deriving from diamino-dianhydro-dideoxy-hexitols (J. Thiem in Makromol. Chemie 187, 2775-2778, 1986). The procedure described therein, however, requires phosgene or phosgene substitutes, which on account of their toxicity necessitate a considerable process-engineering cost and complexity.

Additionally known are polyurethanes of certain monoisocyanates based on 1:4-3:6 dianhydrohexitols (J. Thiem et al., Macromol. Rapid Commun. 19, 21-26, 1998). Homopolymers of this kind, in which the isocyanate unit and the polyol unit are virtually identical, afford no breadth of variation at all in terms of the physical properties, and are therefore not presently in commercial use. Their nature is more that of research compounds, for universities, for example.

Polyurethanes and polyureas formed from certain diisocyanates of 1:4-3:6 dianhydrohexitols and a short-chain diol-1,4-butanediol—and a short-chain diamine-1,4-diaminobutane—and also from 1,4-butanedithiol are known (J. Thiem et al., Macromol. Chem. Phys. 202, 3410-3419, 2001).

Compounds of this kind, however, are not of technical and industrial relevance. The restriction to a single monomer, which, moreover, carries neither ester nor carbonate groups, results in a very restricted breadth of variation of the properties. Consequently they have virtually no applications nowadays.

It was the object of the invention to find new uses of isocyanates based on renewable raw materials.

The object has been achieved in accordance with the claims and the description.

The invention provides for the use of

### A)

2,5-diisocyanato-1,4:3,6-dianhydro-2,5-dideoxy-D-mannitol (I), 2,5-diisocyanato-1,4:3,6-dianhydro-2,5-dideoxy-D-glucitol (II) and/or 2,5-diisocyanato-1,4:3,6-dianhydro-2,5-dideoxy-L-iditol (III) of the formulae

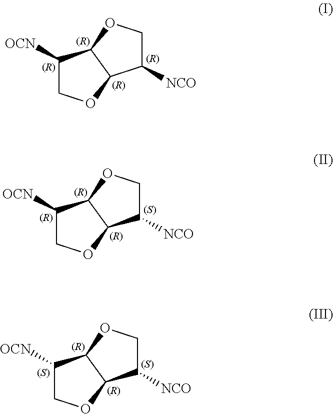

as main component, base component or added component in coating materials, adhesives, varnishes, paints, powder coatings, printing inks, liquid inks, polishes, stains, pigment pastes, masterbatches, filling compounds, sealants, insulants, thermoplastic elastomers, foams, semirigid foams, integral foams, thermal insulants, RIM materials, materials for medical and hygiene applications, fibers, fiber composite components, gels, microcapsules.

The diisocyanato-dianhydro-hexitols (I-III) that are reacted here belong to a group of chemical derivatives composed of what are called renewable raw materials, here more particularly (poly)saccharides, also including, for example, starch (corn starch, potato starch) and sugar (cane sugar, beet sugar). The particular advantage of these compounds and of the application products deriving from them is that they are produced using increasingly less of fossil raw materials such as oil, gas, and coal, thereby reducing CO₂ emissions and hence allowing the start of a transition to a CO₂-neutral mode of production that preserves fossil raw materials. The use, in particular, of diisocyanato-dianhydro-hexitols (I-III) in polyurethanes is opening up a field of use which is large in volume terms. Hence the potential for savings in terms of fossil raw materials is correspondingly high as well.

The compounds of the formulae I-III are used in accordance with the invention as a main component, base component or added component in coating materials (e.g., textile, paper, and leather coating), adhesives, varnishes, paints, powder coatings, printing inks, liquid inks, polishes, stains, pigment pastes, masterbatches, filling compounds, sealants, insulants, thermoplastic elastomers, foams (e.g., slabstock foams, molded foams), semirigid foams (e.g., foam-backed films, energy-absorbing foams, fiber-reinforced foams), integral foams (e.g., rigid and flexible internal foams), thermal insulants, RIM materials, materials for medical and hygiene applications (e.g., wound treatment), fibers, gels, microcapsules.

In general the compounds I-III of the invention are used as co-reactants with compounds possessing at least one reactive functional groups that are reactive toward NCO groups.

The invention therefore also provides for the use of compositions substantially comprising a reaction product of

### A)

2,5-diisocyanato-1,4:3,6-dianhydro-2,5-dideoxy-D-mannitol (I), 2,5-diisocyanato-1,4:3,6-dianhydro-2,5-dideoxy-D-glucitol (II) and/or 2,5-diisocyanato-1,4:3,6-dianhydro-2,5-dideoxy-L-iditol (III), alone or in any desired mixtures, of the formulae

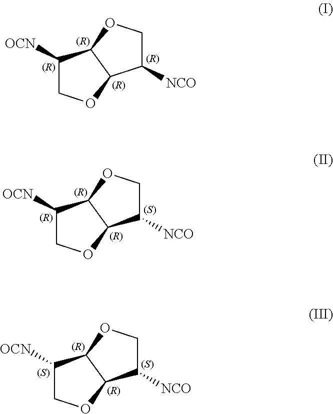

and

### B)

at least one compound having at least one functional group that is reactive toward NCO groups,

the ratio between the NCO component A) and the NCO-reactive component B) containing functional groups, calculated as NCO/NCO-reactive is 0.3:1 to 1.05:1, preferably 0.5:1 to 1:1,

as a main component, base component or added component in coating materials (e.g., textile, paper, and leather coating), adhesives, varnishes, paints, powder coatings, printing inks, liquid inks, polishes, stains, pigment pastes, masterbatches, filling compounds, sealants, insulants, thermoplastic elastomers, foams (e.g., slabstock foams, molded foams), semirigid foams (e.g., foam-backed films, energy-absorbing foams, fiber-reinforced foams), integral foams (e.g., rigid and flexible internal foams), thermal insulants, RIM materials, materials for medical and hygiene applications (e.g., wound treatment), fibers, gels, microcapsules.

Used in accordance with the invention as component A) are 2,5-diisocyanato-1,4:3,6-dianhydro-2,5-dideoxy-D-mannitol (I), 2,5-diisocyanato-1,4:3,6-dianhydro-2,5-dideoxy-D-glucitol (II) and/or 2,5-diisocyanato-1,4:3,6-dianhydro-2,5-dideoxy-L-iditol (III), alone or in any desired mixtures, of the formulae

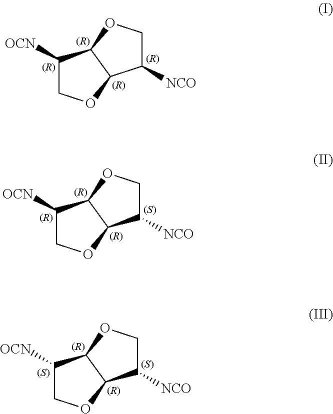

It is possible to use any desired mixtures. Depending on the selection of the compound I-III on the basis of the stereochemistry thereof, certain properties may be obtained in the compositions of the invention, such as, for example, crystallinity (melting point), reactivity, selectivity.

In addition use is also made preferably of oligoisocyanates or polyisocyanates which are preparable from the stated diisocyanates or mixtures thereof by linking by means of urethane, allophanate, urea, biuret, uretdione, amide, isocyanurate, carbodiimide, uretonimine, oxadiazinetrione or iminooxadiazinedione structures. Particularly suitable are isocyanurates and uretdiones.

Component A) of the invention may also be chain-extended.

Chain extenders and optionally monoamines and/or monoalcohols as chain terminators have already been frequently described (EP 0 669 353, EP 0 669 354, DE 30 30 572, EP 0 639 598 or EP 0 803 524). Preference is given to polyesters and polyamines as chain extenders and to monomeric dialcohols as chain terminators.

As a chain extender component it is possible to use polyesters, such as are described later on below.

As chain extender component it is possible to use polyamines having two or more polyisocyanate-reactive amino groups. Suitable polyamines are, for example, adipic dihydrazide, ethylenediamine, diethylenetriamine, triethylenetetramine, tetraethylene-pentamine, pentaethylenehexamine, dipropylenetriamine, hexamethylenediamine, hydrazine, isophoronediamine, N-(2-aminoethyl)-2-aminoethanol, 1,3- and 1,4-phenylenediamine, 4,4′-diphenylmethanediamine, amino-functional polyethylene oxides and/or polypropylene oxides, adducts of salts of 2-acrylamido-2-methylpropane-1-sulfonic acid and ethylenediamine, or any desired combinations of polyamines.

Component A) may also comprise additional di- and polyisocyanates. The di- and polyisocyanates used may consist of any desired aromatic, aliphatic, cycloaliphatic and/or (cyclo)aliphatic di- and/or polyisocyanates.

Suitable aromatic di- or polyisocyanates are in principle all known compounds. Particularly suitable are 1,3- and 1,4-phenylene diisocyanate, 1,5-naphthylene diisocyanate, tolidine diisocyanate, 2,6-tolylene diisocyanate, 2,4-tolylene diisocyanate (2,4-TDI), 2,4′-diphenylmethane diisocyanate (2,4′-MDI), 4,4′-diphenylmethane diisocyanate, mixtures of monomeric diphenylmethane diisocyanates (MDI) and oligomeric diphenylmethane diisocyanates (polymer-MDI), xylylene diisocyanate, tetramethylxylylene diisocyanate and triisocyanatotoluene.

Suitable aliphatic di- or polyisocyanates possess advantageously 3 to 16 carbon atoms, preferably 4 to 12 carbon atoms, in the linear or branched alkylene radical, and suitable cycloaliphatic or (cyclo)aliphatic diisocyanates possess advantageously 4 to 18 carbon atoms, preferably 6 to 15 carbon atoms, in the cycloalkylene radical. (Cyclo)aliphatic diisocyanates are understood sufficiently by the skilled person to involve NCO groups attached both cyclically and aliphatically, as is the case with isophorone diisocyanate, for example. In contrast, cycloaliphatic diisocyanates are understood to be those which have only NCO groups attached directly to the cycloaliphatic ring, e.g., H₁₂MDI. Examples are cyclohexane diisocyanate, methylcyclohexane diisocyanate, ethylcyclohexane diisocyanate, propylcyclohexane diisocyanate, methyldiethylcyclohexane diisocyanate, propane diisocyanate, butane diisocyanate, pentane diisocyanate, hexane diisocyanate, heptane diisocyanate, octane diisocyanate, nonane diisocyanate, nonane triisocyanate, such as 4-isocyanatomethyl-1,8-octane diisocyanate (TIN), decane diisocyanate and triisocyanate, undecane diisocyanate and triisocyanate, dodecane diisocyanates and triisocyanates.

Preference is given to using isophorone diisocyanate (IPDI), hexamethylene diisocyanate (HDI), diisocyanatodicyclohexylmethane (H₁₂MDI), 2-methylpentane diisocyanate (MPDI), 2,2,4-trimethylhexamethylene diisocyanate/2,4,4-trimethylhexamethylene diisocyanate (TMDI), norbornane diisocyanate (NBDI). Very particular preference is given to using IPDI, HDI, TMDI and/or H₁₂MDI, it being also possible with preference to use the isocyanurates and uretdiones.

Likewise suitable are 4-methylcyclohexane 1,3-diisocyanate, 2-butyl-2-ethylpentamethylene diisocyanate, 3(4)-isocyanatomethyl-1-methylcyclohexyl isocyanate, 2-isocyanatopropylcyclohexyl isocyanate, 2,4′-methylenebis(cyclohexyl) diisocyanate, 1,4-diisocyanato-4-methylpentane.

It is of course also possible to use mixtures of the di- and polyisocyanates.

The isocyanates of component A) may be partly or fully blocked. Blocking agents which may be used are all blocking agents. For example, use may be made of phenols such as phenol and p-chlorophenol, alcohols such as benzyl alcohol, oximes such as acetone oxime, methyl ethyl ketoxime, cyclopentanone oxime, cyclohexanone oxime, methyl isobutyl ketoxime, methyl tert-butyl ketoxime, diisopropyl ketoxime, diisobutyl ketoxime or acetophenone oxime, N-hydroxy compounds such as N-hydroxysuccinimide or hydroxypyridines, lactams such as ε-caprolactam, CH-acidic compounds such as ethyl acetoacetate or malonic esters, amines such as diisopropylamine, heterocyclic compounds having at least one heteroatom such as mercaptans, piperidines, piperazines, pyrazoles, imidazoles, triazoles, and tetrazoles, α-hydroxybenzoic esters such as glycolic esters or hydroxamic esters such as benzyl methacrylohydroxamate.

Particularly suitable as blocking agents are acetone oxime, methyl ethyl ketoxime, acetophenone oxime, diisopropylamine, 3,5-dimethylpyrazole, 1,2,4-triazole, ε-caprolactam, butyl glycolate, benzyl methacylohydroxamate or methyl p-hydroxybenzoate.

Suitable in principle as compounds B) are all those having at least one, preferably at least two, functional group(s) reactive toward NCO groups. Suitable functional groups are the following: OH—, NH₂—, NH—, SH—, CH-acidic groups. The compounds B) preferably contain 2 to 4 functional groups. Particularly preferred are alcohol groups and amino groups.

Diamines and polyamines suitable in principle include the following: 1,2-ethylenediamine, 1,2-propylenediamine, 1,3-propylenediamine, 1,2-butylenediamine, 1,3-butylenediamine, 1,4-butylenediamine, 2-(ethylamino)ethylamine, 3-(methylamino)propylamine, 3-(cyclohexylamino)propylamine, 4,4′-diaminodicyclohexylmethane, isophoronediamine, 4,7-dioxadecane-1,10-diamine, N-(2-aminoethyl)-1,2-ethanediamine, N-(3-aminopropyl)-1,3-propanediamine, N,N″-1,2-ethanediylbis(1,3-propanediamine), adipic dihydrazide, diethylenetriamine, triethylenetetramine, tetraethylenepentamine, pentaethylenehexamine, dipropylenetriamine, hydrazine, 1,3- and 1,4-phenylenediamine, 4,4′-diphenylmethanediamine, amino-functional polyethylene oxides and/or polypropylene oxides, adducts of salts of 2-acrylamido-2-methylpropane-1-sulfonic acid, and also hexamethylenediamines, which may also carry one or more C₁-C₄-alkyl radicals. Furthermore, it is also possible to use disecondary or primary/secondary diamines, such as are obtained, for example, in a known way from the corresponding diprimary diamines by reaction with a carbonyl compound, such as a ketone or aldehyde, for example, and by subsequent hydrogenation, or by addition reaction of diprimary diamines with acrylic esters or with maleic acid derivatives.

Mixtures of the stated polyamines can also be used. 1,4-Diaminobutane (1,4-butylenediamine) is used only in mixtures.

Examples that may be given of amino alcohols include monoethanolamine, 3-amino-1-propanol, isopropanolamine, aminoethoxyethanol, N-(2-aminoethyl)ethanolamine, N-ethylethanolamine, N-butylethanolamine, diethanolamine, 3-(hydroxyethylamino)-propanol, and diisopropanolamine, also as mixtures.

CH-acidic compounds. Examples of suitable CH-acidic compounds are derivatives of malonic esters, acetylacetone and/or ethyl acetoacetate.

Suitable as compounds B) are, in particular, all diols and polyols having at least two OH groups that are customarily used in PU chemistry.

Diols and polyols used are ethylene glycol, 1,2-, 1,3-propanediol, diethylene, dipropylene, triethylene, tetraethylene glycol, 1,2-, 1,4-butanediol, 1,3-butylethylpropanediol, 1,3-methylpropanediol, 1,5-pentanediol, bis(1,4-hydroxymethyl)cyclohexane (cyclohexanedimethanol), glycerol, hexanediol, neopentyl|)glycol, trimethylolethane, trimethylolpropane, pentaerythritol, bis-phenol A, B, C, F, norbornylene glycol, 1,4-benzyldimethanol, -ethanol, 2,4-dimethyl-2-ethylhexane-1,3-diol, 1,4- and 2,3-butylene glycol, di-β-hydroxyethylbutanediol, 1,5-pentanediol, 1,6-hexanediol, 1,8-octanediol, decanediol, dodecanediol, neopentylglycol, cyclohexanediol, 3(4),8(9)bis(4-hydroxymethyl)tricyclo[5.2.1.0^{2,6}]decane (Dicidol), 2,2-bis(4-hydroxycyclohexyl)propane, 2,2-bis[4-(β-hydroxyethoxy)phenyl]propane, 2-methylpropane-1,3-diol, 2-methylpentane-1,5-diol, 2,2,4(2,4,4)-trimethylhexane-1,6-diol, hexane-1,2,6-triol, butane-1,2,4-triol, tris(β-hydroxyethyl)isocyanurate, mannitol, sorbitol, polypropylene glycols, polybutylene glycols, xylylene glycol or neopentylglycol hydroxypivalate, hydroxyl acrylates, alone or in mixtures.

Particularly preferred are 1,4-butanediol, 1,2-propanediol, cyclohexanedimethanol, hexanediol, neopentylglycol, decanediol, dodecanediol, trimethylolpropane, ethylene glycol, triethylene glycol, pentane-1,5-diol, hexane-1,6-diol, 3-methylpentane-1,5-diol, neopentylglycol, 2,2,4(2,4,4)-trimethylhexanediol, and neopentylglycol hydroxypivalate. They are used alone or in mixtures. 1,4-Butanediol is used only in mixtures.

Other suitable compounds B) are diols and polyols which contain further functional groups. Preferably they are the hydroxyl-containing polyesters, polycarbonates, polycaprolactones, polyethers, polythioethers, polyesteramides, polyacrylates, polyvinyl alcohols, polyurethanes or polyacetals, which are known per se and are linear or have a low degree of branching. They preferably have a number-average molecular weight of 134 to 20 000 g/mol, more preferably 134-4000 g/mol. The hydroxyl-containing polymers used are preferably polyesters, polyethers, polyacrylates, polyurethanes, polyvinyl alcohols and/or polycarbonates having an OH number of 5-500 (in mg of KOH/gram).

Preference is given to hydroxyl-containing polyesters—polyester polyols—that are linear or have a low degree of branching, or to mixtures of such polyesters. They are prepared, for example, by reaction of diols with substoichiometric amounts of dicarboxylic acids, corresponding dicarboxylic anhydrides, corresponding dicarboxylic esters of lower alcohols, lactones or hydroxycarboxylic acids.

Diols and polyols suitable for preparing the preferred polyester polyols, in addition to the diols and polyols stated above, are also 2-methylpropanediol, 2,2-dimethylpropanediol, diethylene glycol, dodecane-1,12-diol, 1,4-cyclohexanedimethanol, and 1,2- and 1,4-cyclohexanediol.

Preference is given to using 1,4-butanediol, 1,2-propanediol, cyclohexanedimethanol, hexanediol, neopentylglycol, decanediol, dodecanediol, trimethylolpropane, ethylene glycol, triethylene glycol, 1,5-pentanediol, 1,6-hexanediol, 3-methylpentane-1,5-diol, neopentylglycol, 2,2,4 (2,4,4)-trimethylhexanediol, and neopentylglycol hydroxypivalate for preparing the polyester polyols.

Dicarboxylic acids or derivatives that are suitable for preparing the polyester polyols may be aliphatic, cycloaliphatic, aromatic and/or heteroaromatic in nature and may optionally be substituted, by halogen atoms, for example, and/or unsaturated.

The preferred dicarboxylic acids or derivatives include succinic, adipic, suberic, azelaic, and sebacic acid, 2,2,4 (2,4,4)-trimethyladipic acid, phthalic acid, phthalic anhydride, isophthalic acid, terephthalic acid, dimethyl terephthalate, tetrahydrophthalic acid, maleic acid, maleic anhydride, and dimeric fatty acids.

Suitable polyester polyols are also those which can be prepared in a known way by ring opening from lactones, such as—caprolactone, and simple diols as starter molecules. Monoesters and polyesters formed from lactones as well, e.g., from ε-caprolactone or hydroxycarboxylic acids, e.g., hydroxypivalic acid, ε-hydroxydecanoic acid, ε-hydroxycaproic acid, thioglycolic acid, can be used as starting materials for preparing the polymers G). Polyesters from the polycarboxylic acids stated above (page 6) and/or derivatives thereof and from polyphenols, such as hydroquinone, bisphenol-A, 4,4′-dihydroxybiphenyl or bis(4-hydroxyphenyl) sulfone; polyesters of carbonic acid which are obtainable from hydroquinone, diphenylolpropane, p-xylylene glycol, ethylene glycol, butanediol or hexane-1,6-diol and other polyols by customary condensation reactions, e.g. with phosgene or diethyl and/or diphenyl carbonate, or from cyclic carbonates, such as glycol carbonate or vinylidene carbonate, by polymerization in a known way; polyesters of silicic acid, polyesters of phosphoric acid, e.g., from methane-, ethane-, β-chloroethane-, benzene- or styrenephosphoric acid or derivatives thereof, such as phosphoric acid chlorides or phosphoric esters, for example, and polyalcohols or polyphenols of the type specified above; polyesters of boric acid; polysiloxanes, such as the products, for example, obtainable by hydrolysis of dialkyldichlorosilanes with water and subsequent treatment with polyalcohols, the products obtainable by addition reaction of polysiloxane dihydrides with olefins, such as allyl alcohol or acrylic acid, are suitable as starting materials for the preparation of the compounds B).

The polyesters can be obtained in a conventional way by condensation in an inert-gas atmosphere at temperatures from 100 to 260° C., preferably 130 to 220° C., in the melt or in an azeotropic regime, as is described, for example, in Methoden der Organischen Chemie (Houben-Weyl); volume 14/2, pages 1 to 5, 21 to 23, 40 to 44, Georg Thieme Verlag, Stuttgart, 1963, or in C. R. Martens, Alkyd Resins, pages 51 to 59, Reinhold Plastics Appl. Series, Reinhold Publishing Comp., New York, 1961.

Likewise possible for use with preference are (meth)acrylates and poly(meth)acrylates containing OH groups. They are prepared by the copolymerization of (meth)acrylates, where certain components carry OH groups while others do not. Accordingly, a randomly distributed polymer containing OH groups is produced that carries none, one or a large number of OH group(s). Polymers of this kind are described in

High solids hydroxy acrylics with tightly controlled molecular weight. van Leeuwen, Ben., SC Johnson Polymer, Neth. PPCJ, Polymers Paint Colour Journal (1997), 187(4392), 11-13;

Special techniques for synthesis of high solid resins and applications in surface coatings. Chakrabarti, Suhas; Ray, Somnath., Berger Paints India Ltd., Howrah, India. Paintindia (2003), 53(1), 33-34,36,38-40;

VOC protocols and high solid acrylic coatings. Chattopadhyay, Dipak K.; Narayan, Ramanuj; Raju, K. V. S. N. Organic Coatings and Polymers Division, Indian Institute of Chemical Technology, Hyderabad, India. Paintindia (2001), 51(10), 31-42.

The diols and dicarboxylic acids, and/or derivatives thereof, that are used for preparing the polyester polyols can be employed in any desired mixtures.

It is also possible to use mixtures of polyester polyols and diols. Suitable compounds B) are also the reaction products of polycarboxylic acids and glycidyl compounds, as are described in DE-A 24 10 513, for example.

Examples of glycidyl compounds which can be used are esters of 2,3-epoxy-1-propanol with monobasic acids having 4 to 18 carbon atoms, such as glycidyl palmitate, glycidyl laurate, and glycidyl stearate, alkylene oxides having 4 to 18 carbon atoms, such as butylene oxide, and glycidyl ethers, such as octyl glycidyl ether.

Compounds B) are also compounds which as well as an epoxide group also carry at least one further functional group, such as, for example, carboxyl, hydroxyl, mercapto or amino groups, capable of reaction with an isocyanate group. Particularly preferred are 2,3-epoxy-1-propanol and epoxidized soybean oil.

It is possible to use any desired combinations of the compounds B).

### Preparation

The reaction of components A) and B) may be carried out in suitable assemblies, stirred tanks, static mixers, tube reactors, kneading devices, extruders or other reaction spaces with or without a mixing function. The reaction is carried out at temperatures between room temperature and 220° C., preferably between 40° C. and 120° C., and lasts for between a few hours and several weeks, depending on temperature and reaction components A) and B). A reaction time of between 30 min and 24 h is preferred. The ratio between the NCO component A) and the NCO-reactive functional groups-containing component B), calculated as NCO/NCO-reactive, is 0.3:1 to 1.05:1, preferably 0.5:1 to 1:1. The end product possesses no notable free NCO groups (<0.5% by weight).

To accelerate the polyaddition reaction it is possible to use the catalysts that are customary in PU chemistry. They are used at a concentration of 0.001% to 2% by weight, preferably of 0.01% to 0.5% by weight, based on the reaction components employed. Catalysts are, for example, tertiary amines such as triethylamine, pyridine or N,N-dimethylaminocyclohexane or metal salts such as iron(III) chloride, molybdenum glycolate, and zinc chloride. Tin(II) and (IV) compounds have proven particularly suitable. Particular mention may be made here of dibutyltin dilaurate (DBTL) and tin octoate.

The compositions of the invention may be present in solid, viscous, liquid, and also powder form.

Furthermore, the compositions may also comprise auxiliaries and additives, selected from inhibitors, organic solvents, which optionally contain unsaturated moieties, interface-active substances, oxygen scavengers and/or free-radical scavengers, catalysts, light stabilizers, color brighteners, photoinitiators, photosensitizers, thixotropic agents, antiskinning agents, defoamers, dyes, pigments, fillers, and matting agents. The amount varies greatly as a function of the field of use and nature of the auxiliary and additive.

Organic solvents contemplated include all liquid substances which do not react with other ingredients, examples being acetone, ethyl acetate, butyl acetate, xylene, Solvesso 100, Solvesso 150, methoxypropyl acetate, and Dibasic ester.

Likewise it is possible for the customary additives, such as flow-control agents, e.g., polysilicones or acrylates, light stabilizers, e.g. sterically hindered amines, or other auxiliaries, as described in EP 0 669 353, for example, to be added in a total amount of 0.05% to 5% by weight. Fillers and pigments such as titanium dioxide, for example, can be added in an amount of up to 50% by weight of the overall composition.

The present invention provides in one preferred aspect for the use of the isocyanates of the invention of the formulae I to III as coating materials, more particularly as primer, tiecoat, topcoat, clearcoat, adhesive or sealing material, and also provides the coating materials themselves, with particular preference with compounds of component B).

The invention also provides for the use of the isocyanates of the invention for producing coatings in liquid and powder form on metal, plastics, glass, wood, textile, MDF (Middle Density Fiber Boards) or leather substrates.

The invention also provides for the use of the isocyanates of the invention as adhesive compositions for adhesive bonds of metal, plastics, glass, wood, textile, paper, MDF (Middle Density Fiber Boards) or leather substrates.

Likewise provided by the invention are metal-coating compositions, more particularly for automobile bodies, motor and pedal cycles, architectural components, and household appliances, wood-coating compositions, glass-coating compositions, textile-coating compositions, leather-coating compositions, and plastics-coating compositions which comprise the isocyanates of the invention.

The coating may either be used alone, or may be one coat in a multicoat system. It may be applied, for example, as a primer, as a tiecoat or as a topcoat or clearcoat. The coats situated above or below the coating may be cured either conventionally, thermally, or else, alternatively, by radiation.

1. A material, comprising, in at least partially polymerized form:
2,5-diisocyanato-1,4:3,6-dianhydro-2,5-dideoxy-D-mannitol of formula (I) 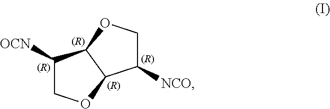
2,5-diisocyanato-1,4:3,6-dianhydro-2,5-dideoxy-D-glucitol of formula (II) 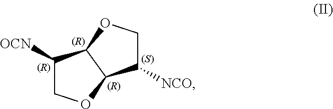
and 2,5-diisocyanato-1,4:3,6-dianhydro-2,5-dideoxy-L-iditol of formula (III) 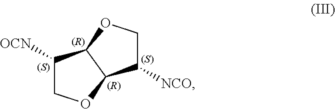
wherein the material is at least one selected from the group consisting of a coating material, an adhesive, a varnish, a paint, a powder coating, a printing ink, a liquid ink, a polish, a stain, a pigment paste, a masterbatch, a filling compound, a sealant, an insulant, a thermoplastic elastomer, a foam, a semirigid foam, an integral foam, a thermal insulant, a RIM material, a medical or hygiene material, a fiber, a fiber composite component, a gel, and a microcapsule.

2. A process for producing the material of claim 1, the process comprising reacting a component (A) comprising at least one isocyanate selected from the group consisting of
2,5-diisocyanato-1,4:3,6-dianhydro-2,5-dideoxy-D-mannitol of formula (I), 2,5-diisocyanato-1,4:3,6-dianhydro-2,5-dideoxy-D-glucitol of formula (II), and 2,5-diisocyanato-1,4:3,6-dianhydro-2,5-dideoxy-L-iditol of formula (III) 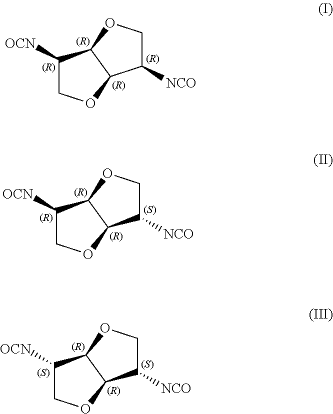
with an NCO-reactive component (B) comprising a compound comprising a functional group that is reactive toward an NCO group, to obtain a reaction product,
wherein a ratio between a NCO component (A) and a NCO-reactive component (B), calculated as NCO/NCO-reactive, is 0.3:1 to 1.05:1.

3. The process of claim 2, wherein component (A) further comprises an oligoisocyanate or a polyisocyanate.

4. The process of claim 2, wherein component (A) is chain-extended.

5. The process of claim 2, wherein component (A) is blocked.

6. The process of claim 2, wherein component (A) further comprises at least one selected from the group consisting of an aromatic diisocyanate, an aliphatic diisocyanate, a cycloaliphatic diisocyanate, a (cyclo)aliphatic diisocyanate, an aromatic polyisocyanate, an aliphatic polyisocyanate, a cycloaliphatic polyisocyanate, and a (cyclo)aliphatic polyisocyanate.

7. The process of claim 2, wherein component (A) further comprises at least one selected from the group consisting of 1,3-phenylene diisocyanate, 1,4-phenylene diisocyanate, 1,5-naphthylene diisocyanate, tolidine diisocyanate, 2,6-tolylene diisocyanate, 2,4-tolylene diisocyanate (2,4-TDI), 2,4′-diphenylmethane diisocyanate (2,4′-MDI), 4,4′-diphenylmethane diisocyanate, a mixture comprising a monomeric diphenylmethane diisocyanate (MDI) and an oligomeric diphenylmethane diisocyanate (polymeric MDI), xylylene diisocyanate, tetramethylxylylene diisocyanate, and triisocyanatotoluene.

8. The process of claim 2, wherein component (A) further comprises at least one selected from the group consisting of isophorone diisocyanate (IPDI), hexamethylene diisocyanate (HDI), diisocyanatodicyclohexylmethane (H₁₂MDI), 2-methylpentane diisocyanate (MPDI), 2,2,4-trimethylhexamethylene diisocyanate (2,2,4-TMDI), 2,4,4-trimethylhexamethylene diisocyanate (2,4,4-TMDI), and norbornane diisocyanate (NBDI).

9. The process of claim 2, wherein component (B) comprises a compound comprising at least two functional groups that are reactive toward an NCO group, wherein the reactive functional groups are at least one selected from the group consisting of an OH—, a NH₂—, a NH—, a SH—, and a CH-acidic group.

10. The process of claim 2, wherein component (B) comprises at least one selected from the group consisting of 1,2-ethylenediamine, 1,2-propylenediamine, 1,3-propylenediamine, 1,2-butylenediamine, 1,3-butylenediamine, 1,4-butylenediamine, 2-(ethylamino)ethylamine, 3-(methylamino)propylamine, 3-(cyclohexylamino)propylamine, 4,4′-diaminodicyclohexylmethane, isophoronediamine, 4,7-dioxadecane-1,10-diamine, N-(2-aminoethyl)-1,2-ethanediamine, N-(3-aminopropyl)-1,3-propanediamine, N,N″-1,2-ethanediylbis(1,3-propanediamine), adipic dihydrazide, diethylenetriamine, triethylenetetramine, tetraethylenepentamine, pentaethylenehexamine, dipropylenetriamine, hydrazine, 1,3- and 1,4-phenylenediamine, 4,4′-diphenylmethanediamine, an amino-functional polyethylene oxide, an amino-functional polypropylene oxide, an adduct of a salt of 2-acrylamido-2-methylpropane-1-sulfonic acid, and a hexamethylenediamine, which may comprise a C₁-C₄-alkyl radical.

11. The process of claim 2, wherein component (B) comprises at least one selected from the group consisting of monoethanolamine, 3-amino-1-propanol, isopropanolamine, aminoethoxyethanol, N-(2-aminoethyl)ethanolamine, N-ethylethanolamine, N-butylethanolamine, diethanolamine, 3-(hydroxyethylamino)-1-propanol, and diisopropanolamine.

12. The process of claim 2, wherein component (B) comprises at least one selected from the group consisting of a derivative of a malonic ester, acetylacetone, and ethyl acetoacetate.

13. The process of claim 2, wherein component (B) comprises at least one selected from the group consisting of ethylene glycol, 1,2-propanediol, 1,3-propanediol, diethylene, dipropylene, triethylene, tetraethylene glycol, 1,2-butanediol, 1,4-butanediol, 1,3-butylethylpropanediol, 1,3-methylpropanediol, 1,5-pentanediol, bis(1,4-hydroxymethyl)cyclohexane, glycerol, hexanediol, neopentylglycol, trimethylolethane, trimethylolpropane, pentaerythritol, bis-phenol A, bis-phenol B, bis-phenol C, bis-phenol F, norbornylene glycol, 1,4-benzyldimethanol, 1,4-benzyldiethanol, 2,4-dimethyl-2-ethylhexane-1,3-diol, 1,4-butylene glycol, 2,3-butylene glycol, di-β-hydroxyethylbutanediol, 1,5-pentanediol, 1,6-hexanediol, 1,8-octanediol, decanediol, dodecanediol, neopentylglycol, cyclohexanediol, 3(4)bis(4-hydroxymethyl)tricyclodecane, 8(9)bis(4-hydroxymethyl)tricyclodecane, 2,2-bis(4-hydroxycyclohexyl)propane, 2,2-bis[4-(β-hydroxyethoxy)phenyl]propane, 2-methylpropane-1,3-diol, 2-methylpentane-1,5-diol, (2,4,4)-trimethylhexane-1,6-diol, (2,2,4)-trimethylhexane-1,6-diol, hexane-1,2,6-triol, butane-1,2,4-triol, tris(β-hydroxyethyl)isocyanurate, mannitol, sorbitol, a polypropylene glycol, a polybutylene glycol, xylylene glycol, neopentylglycol hydroxypivalate, and a hydroxyl acrylate.

14. The process of claim 2, wherein component (B) is at least one selected from the group consisting of 1,4-butanediol, 1,2-propanediol, cyclohexanedimethanol, hexanediol, neopentylglycol, decanediol, dodecanediol, trimethylolpropane, ethylene glycol, triethylene glycol, pentane-1,5-diol, hexane-1,6-diol, 3-methylpentane-1,5-diol, neopentylglycol, 2,2,4(2,4,4)-trimethylhexanediol, and neopentylglycol hydroxypivalate.

15. The process of claim 2, wherein component (B) is at least one selected from the group consisting of a diol and a polyol which comprise a further functional group.

16. The process of claim 2, wherein component (B) is a polyester polyol obtained by a process comprising reacting at least one selected from the group consisting of 1,4-butanediol, 1,2-propanediol, cyclohexanedimethanol, hexanediol, neopentylglycol, decanediol, dodecanediol, trimethylolpropane, ethylene glycol, triethylene glycol, pentane-1,5-diol, hexane-1,6-diol, 3 methylpentane-1,5-diol, neopentylglycol, 2,2,4 (2,4,4)-trimethylhexanediol, and neopentylglycol hydroxypivalate
with
at least one selected from the group consisting of a succinic acid, adipic acid, suberic acid, azelaic acid, sebacic acid, 2,2,4 (2,4,4)-trimethyladipic acid, phthalic acid, phthalic anhydride, isophthalic acid, terephthalic acid, dimethyl terephthalate, tetrahydrophthalic acid, maleic acid, maleic anhydride, and a dimeric fatty acid.

17. The process of claim 2, wherein component (B) comprises at least one hydroxyl-comprising polymer selected from the group consisting of a polyester, a polyether, a polyacrylate, a polyurethane, a polyvinyl alcohols alcohol, and a polycarbonate, wherein the polymer has an OH number of 5-500 (in mg of KOH/gram).

18. The process of claim 2, wherein component (B) is at least one selected from the group consisting of a (meth)acrylate and a poly(meth)acrylate comprising an OH group.

19. The process of claim 2, wherein component (B) comprises a reaction product obtained by a process comprising reacting a polycarboxylic acid with a glycidyl compound.

20. The process of claim 2, wherein component (B) comprises a compound comprising an epoxide group and a further functional group, which is capable of reacting with an isocyanate group.

21. A process for producing a coating composition, the process comprising combining a product obtained by reacting an isocyanate of formula I, (II), or (III) 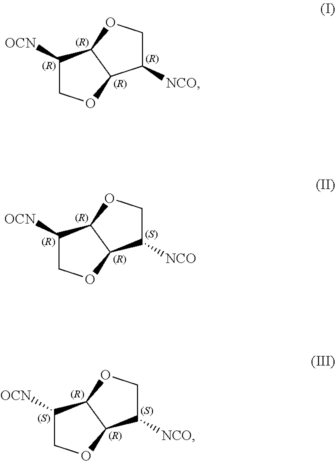
with a compound comprising a functional group reactive with an isocyanate, with a coating material.

22. The process of claim 21, wherein the coating material is a primer, a tiecoat, a topcoat, a clearcoat, or a sealing material.

23. The process of claim 21, wherein the coating material is a coating composition or adhesive composition suitable for metal, plastic, wood, textiles, glass, leather, MDF (Medium Density Fiberboard).

24. A coating material, comprising;
at least one isocyanate selected from the group consisting of 2,5-diisocyanato-1,4:3,6-dianhydro-2,5-dideoxy-D-mannitol of formula (I) 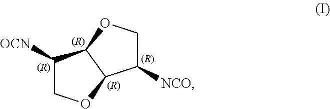
2,5-diisocyanato-1,4:3,6-dianhydro-2,5-dideoxy-D-glucitol of formula (II) 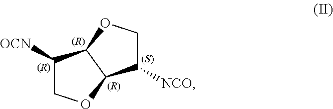
and 2,5-diisocyanato-1,4:3,6-dianhydro-2,5-dideoxy-L-iditol of formula (III).

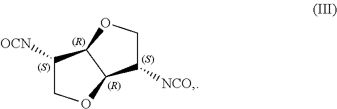

25. The material of claim 24, in the form of a metal-coating composition, a wood-coating composition, a leather-coating composition, a textile-coating composition, a plastic-coating composition, or a glass-coating composition.

26. The metal-coating composition of claim 25, wherein the coating is suitable for an automobile body, a motor cycle, a pedal cycle, an architectural component, and a household appliance.
